(19) United States
(12) Patent Application Publication
(10) Pub. No.: US 2019/0219655 A1
(43) Pub. Date: Jul. 18, 2019

(54) METHOD FOR POST-PROCESSING IMAGES FOR COMPENSATING RESPIRATORY MOVEMENTS

(71) Applicants: INSTITUT NATIONAL DE LA SANTE ET DE LA RECHERCHE MEDICALE (INSERM), Paris (FR); UNIVERSITÉ PARIS DIDEROT - PARIS 7, Paris (FR); CENTRE NATIONAL DE LA RECHERCHE SCIENTIFIQUE, Paris (FR); ASSISTANCE PUBLIQUE ? HÔPITAUX DE PARIS, Paris (FR)

(72) Inventors: Benjamin LEPORQ, VILLEURBANNE CEDEX (FR); Jean - Luc DAIRE, PARIS (FR); Bernard VAN BEERS, PARIS (FR)

(21) Appl. No.: 16/332,321

(22) PCT Filed: Sep. 13, 2017
(86) PCT No.: PCT/EP2017/073016

§ 371 Date: Mar. 11, 2019

(30) Foreign Application Priority Data
Sep. 13, 2016 (EP) 16306159.1

(51) Int. Cl. G01R 33/565; G01R 33/56; A61B 5/055; A61B 5/00; G06T 7/70; G06T 7/00

(52) U.S. Cl. CPC G01R 33/56509; G01R 33/5601; G01R 33/5608; A61B 5/055; G06T 2207/10088; G06T 7/70; G06T 7/0014; G06T 2207/30061; G06T 2207/30056; A61B 5/7207

(57) ABSTRACT

Disclosed is the use of magnetic resonance imaging in the medical field. One issue addressed is the compensating of respiratory movements in the obtained images with magnetic resonance imaging. For this, the method proposes to choose a reference image in the initial set, the determined position for the reference image being a reference position and to compensate the difference between the determined position and the reference position to obtain a corrected set of images for each image of the initial set. Such method can be implemented in a computer and may be used to provide additional functionalities to magnetic resonance imager and renders the taking of images by a magnetic resonance imager easier.

### TECHNICAL FIELD OF THE INVENTION

The present invention concerns a method for post-processing a set of images. The present invention also relates to an associated computer program product and computer readable medium.

### BACKGROUND OF THE INVENTION

Magnetic resonance imaging (MRI), nuclear magnetic resonance imaging (NMRI), or magnetic resonance tomography (MRT) is a medical imaging technique used in radiology to image the anatomy and the physiological processes of the body in both health and disease. MRI scanners use strong magnetic fields, radio waves, and field gradients to form images of the body.

MRI is based upon the science of Nuclear Magnetic Resonance (NMR). Certain atomic nuclei can absorb and emit radio frequency energy when placed in an external magnetic field. In clinical and research MRI, hydrogen atoms are most-often used to generate a detectable radio-frequency signal that is received by antennas in close proximity to the anatomy being examined. Hydrogen atoms exist naturally in people and other biological organisms in abundance, particularly in water and fat. For this reason, most MRI scans essentially map the location of water and fat in the body. Pulses of radio waves are used to excite the nuclear spin energy transition, and magnetic field gradients localize the signal in space. By varying the parameters of the pulse sequence, different contrasts can be generated between tissues based on the relaxation properties of the hydrogen atoms therein. Since its early development in the 1970s and 1980s, MRI has proven to be a highly versatile imaging technique. While MRI is most prominently used in diagnostic medicine and biomedical research, it can also be used to form images of non-living objects. MRI scans are capable of producing a variety of chemical and physical data, in addition to detailed spatial images.

However, magnetic resonance imaging of living tissues is an imaging technique requiring relatively long acquisition periods of time to ensure that a sufficient image resolution is obtained. This raises an issue in the case of living subjects such as animals or humans for which most organs are subject to internal movements of the subject. The internal movements result from several biological phenomena. The cardiovascular and respiratory activities of the subject are two examples of such biological phenomena.

Such issue is all the more sensitive when it is desired to monitor the temporal evolution of a given quantity in a region of interest for the subject. Furthermore, such issue may be enhanced by the use of a contrast agent since the concentration of the contrast agent, and thus the intensity of the measured signal, varies rapidly.

It is therefore desired to be able to compensate the internal movements of the subject in a set of MRI images. Notably, the respiratory movement is a key issue because of its relatively large amplitude (about 5 centimeters for a human).

One simple way to reduce the respiratory movement is to prevent it. More precisely, the subject is required to hold his breath during acquisition.

However, such method is not applicable for animals or babies. In addition, the method is not applicable for total imaging times longer than 5 to 10 seconds. Furthermore, such method does not circumvent the issue of the reflex movement of the diaphragm.

It is also known from the document Higgins C. B., de Roos A., 2006, MRI and CT of the Cardiovascular System, chapter 18, another method in which the acquisition of an image is triggered when a specific part of the organ, for example, an interface with another close organ, crosses a predetermined trigger area. The detection that the interface crosses the predetermined trigger area is achieved by monitoring the intensity value of a group of pixel, the detection being triggered when the monitored intensity is superior to a threshold value.

Nevertheless, such method is not operable in case the threshold value evolves with time. For instance, such method is not compatible with an enhanced MRI imaging technique.

Other methods are known from the article by White M. J. et al. whose title is “Diaphragm alignment of multiple breath-hold dynamic contrast-enhanced MRI of the liver for quantitative parameter estimation” (Proc. Intl. Soc. Mag. Reson. Med. 11 (2004)) and the documents US 2015/310299 A1 and U.S. Pat. No. 5,613,492 A.

However, none of these methods enable to provide accurate corrections with an easy implementation.

### SUMMARY OF THE INVENTION

The invention aims at proposing a method for processing a set of images which alleviates the previous drawbacks.

To this end, the invention concerns a method for post-processing a set of images, the method comprising the step of providing an initial set of images, the initial set of images comprising a plurality of images of a region of interest of a subject having a diaphragm, the images defining a field of view, the field of view including the region of interest and a part of the diaphragm of the subject. For each image of the initial set, the method also comprises a step of determining the position of the diaphragm of the subject to obtain a determined position for the considered image. The method also comprises a step of choosing a reference image in the initial set, the determined position for the reference image being a reference position, and, for each image of the initial set, a step of compensating the difference between the determined position and the reference position to obtain a corrected set of images.

Thanks to the invention, it is possible to provide a corrected set of images of the region of interest, in which at least part of the respiratory activity movements of the subject has been removed. This notably enables to monitor the temporal evolution of a given physical quantity in the region of interest.

According to further aspects of the invention, which are advantageous but not compulsory, the method may include one or several of the following features, taken in any technically admissible combination:
the field of view includes the whole diaphragm.
the field of view includes a portion of at least one fourth of the lung.
the region of interest is the liver.
each image is a map of a signal intensity on the field of view, the step of determining comprising the operation of calculating a spatial derivative of the signal intensity along at least one detection line, to obtained a calculated derivative, the operation of obtaining the position of the extremum of the derivative and the operation of deducing the position of a point belonging to the diaphragm based on the obtained extremum.
the calculating operation is achieved for at least 10 detection lines of one image.
a craniocaudal axis is defined for the subject, the or each detection line extending on each image in a direction parallel to the craniocaudal axis.
the calculating and obtaining operations are carried out for several distinct detection lines, to obtain several positions, the deducing operation being carried out by calculating an average of the obtained positions.
the compensating step is achieved by using a circular permutation.
each image comprises voxels, the circular permutation being applied to a set of non null voxels arranged along a given direction.
the time interval between two images of the initial set of images is comprised between 2 seconds and 5 seconds.
the images have been acquired with a quantitative imaging technique.
the quantitative imaging technique is a magnetic resonance imaging technique.
the magnetic resonance imaging technique includes the use of a contrast agent

which is injected before the acquisition of the initial set of images.

The specification also relates to a computer program product comprising instructions for carrying out the steps of a method as previously described when said computer program product is executed on a suitable computer device.

The specification also concerns a computer readable medium having encoded thereon a computer program as previously described.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 shows schematically a system and a computer program product whose interaction enables to carry out a method for post-processing;

FIG. 2 shows a flowchart of the method for post-processing images; and

FIGS. 3 to 5 illustrate the results obtained by an example of experiment corresponding to the carrying out of the method for post-processing.

### DETAILED DESCRIPTION OF SOME EMBODIMENTS

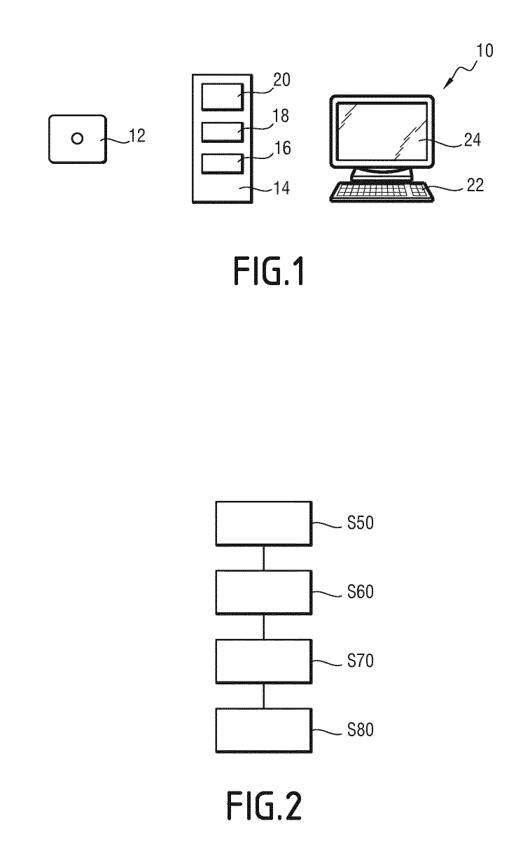

A system 10 and a computer program product 12 are represented in FIG. 1. The interaction between the computer program product 12 and the system 10 enables to carry out a method for post-processing images.

System 10 is a computer. In the present case, system 10 is a laptop.

More generally, system 10 is a computer or computing system, or similar electronic computing device adapted to manipulate and/or transform data represented as physical, such as electronic, quantities within the computing system's registers and/or memories into other data similarly represented as physical quantities within the computing system's memories, registers or other such information storage, transmission or display devices.

System 10 comprises a processor 14, a keyboard 22 and a display unit 24.

The processor 14 comprises a data-processing unit 16, memories 18 and a reader 20. The reader 20 is adapted to read a computer readable medium.

The computer program product 12 comprises a computer readable medium.

The computer readable medium is a medium that can be read by the reader of the processor. The computer readable medium is a medium suitable for storing electronic instructions, and capable of being coupled to a computer system bus.

Such computer readable storage medium is, for instance, a disk, a floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

A computer program is stored in the computer readable storage medium. The computer program comprises one or more stored sequence of program instructions.

The computer program is loadable into the data-processing unit and adapted to cause execution of the method for post-processing images when the computer program is run by the data-processing unit.

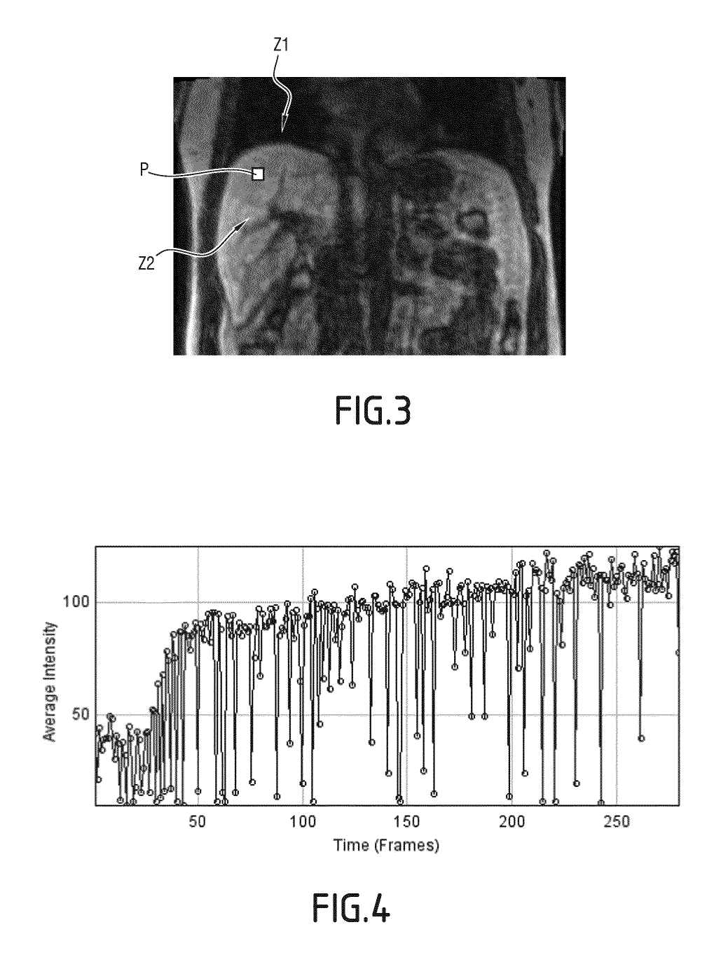

Operation of the system 10 is now described by illustrating an example of carrying out the method for post-processing images as illustrated by the flowchart of FIG. 2.

The method for post-processing a set of images is used to convert an initial set of images in another set of images.

The method for post-processing enables to obtain a set of images in which the respiratory motion is compensated.

According to the example of FIG. 2, the method comprises a step of providing S50, a step of determining S60, a step of choosing S70 and a step of compensating S80.

At the step of providing S50, an initial set of images is provided.

The initial set of image comprises a plurality of images of a region of interest of a subject.

The region of interest encompasses a least a part of the liver.

According a specific embodiment, the region of interest includes whole the liver.

The subject is, for instance, an animal, such as a mammal.

Mice, rats, and more generally small animals are examples of such subjects.

According to another embodiment, the subject is a human being.

In any case, the subject has a diaphragm.

In human anatomy, the thoracic diaphragm, or simply the diaphragm, is a sheet of internal skeletal muscle that extends across the bottom of the thoracic cavity. The diaphragm separates the thoracic cavity containing the heart and lungs, from the abdominal cavity and performs an important function in respiration: as the diaphragm contracts, the volume of the thoracic cavity increases and air is drawn into the lungs.

The term “diaphragm” in anatomy can refer to other flat structures such as the urogenital diaphragm or pelvic diaphragm, but “the diaphragm” generally refers to the thoracic diaphragm. In humans, the diaphragm is slightly asymmetric—its right half is higher up (superior) to the left half, since the large liver rests beneath the right half of the diaphragm.

Other mammals have diaphragms, and other vertebrates such as amphibians and reptiles have diaphragm-like structures, but important details of the anatomy vary, such as the position of lungs in the abdominal cavity.

The images of the set of images are acquired with a magnetic resonance imaging technique.

According to the specific example, the magnetic resonance imaging technique includes the use of a contrast agent which is injected before the acquisition of the initial set of images.

The magnetic resonance imaging technique is thus enhanced by a contrast agent

MRI contrast agents are a group of contrast media used to improve the visibility of internal body structures in magnetic resonance imaging (MRI). The most commonly used compounds for contrast enhancement are gadolinium-based. Such MRI contrast agents shorten the relaxation times of atoms within body tissues following oral or intravenous administration. In MRI scanners, sections of the body are exposed to a very strong magnetic field causing primarily the hydrogen nuclei (“spins”) of water in tissues to be polarized in the direction of the magnetic field. An intense radiofrequency pulse is applied that tips the magnetization generated by the hydrogen nuclei in the direction of the receiver coil where the spin polarization can be detected. Random molecular rotational oscillations matching the resonance frequency of the nuclear spins provide the “relaxation” mechanisms that bring the net magnetization back to its equilibrium position in alignment with the applied magnetic field. The magnitude of the spin polarization detected by the receiver is used to form the MR image but decays with a characteristic time constant known as the T1 relaxation time. Water protons in different tissues have different T1 values, which is one of the main sources of contrast in MR images. A contrast agent usually shortens, but in some instances increases, the value of T1 of nearby water protons thereby altering the contrast in the image.

Most clinically used MRI contrast agents work by shortening the T1 relaxation time of protons inside tissues via interactions with the nearby contrast agent. Thermally driven motions of the strongly paramagnetic metal ions in the contrast agent generate the oscillating magnetic fields that provide the relaxation mechanisms that enhance the rate of decay of the induced polarization. The systematic sampling of this polarization over the spatial region of the tissue being examined forms the basis for construction of the image.

MRI contrast agents may be administered by injection into the blood stream or orally, depending on the subject of interest. Oral administration is well suited to G.I. tract scans, while intravascular administration proves more useful for most other scans. A variety of agents of both types enhances scans routinely.

For instance, the contrast agent is gadoxetate.

All paramagnetic agent used as contrast agent may be considered in this context.

The magnetic resonance imaging technique involves successive frames of a dynamic MRI acquisition.

According to the specific embodiment described, the magnetic resonance imaging technique involves successive echoes of a multiple-gradient echo sequence and the multiple-gradient echo sequence is a spoiled gradient echo sequence.

In addition, the magnetic resonance imaging technique is carried out by a clinical system operating at magnetic field with a magnitude of 3.0 Tesla (T).

The time interval between two images of the initial set of images is comprised between 2 seconds and 5 seconds.

Each image associates to each pixel of the image the amplitude of the measured signal in the magnetic resonance imaging technique and the phase of the measured signal in the magnetic resonance imaging technique.

In other words, for each image, it can be defined a magnitude map and a phase map.

In addition, during the acquisition, the image defines a field of view.

The image is a map of a signal intensity on the field of view.

The field of view includes the region of interest and a part of the diaphragm of the subject.

The diaphragm is the interface between the lung and the liver.

The lungs are the primary organs of respiration in humans and many other animals including a few fish and some snails. In mammals and most other vertebrates, two lungs are located near the backbone on either side of the heart. Their function in the respiratory system is to extract oxygen from the atmosphere and transfer it into the bloodstream, and to release carbon dioxide from the bloodstream into the atmosphere, in a process of gas exchange. Respiration is driven by different muscular systems in different species. Mammals, reptiles and birds use their musculoskeletal systems to support and foster breathing. In early tetrapods, air was driven into the lungs by the pharyngeal muscles via buccal pumping, a mechanism still seen in amphibians. In humans, the primary muscle that drives breathing is the diaphragm. The lungs also provide airflow that makes vocal sounds including human speech possible.

According to a specific embodiment, the field of view includes the whole diaphragm.

According to another embodiment and the field of view includes a portion of at least one fourth of the lung.

The portion is evaluated by calculating the ratio of the area of the portion of the lung in the field of view and the total area of the lung. Such area corresponds to the area in cross-section imaging.

The step of providing S50 is, for instance, made by providing a file comprising the set of images.

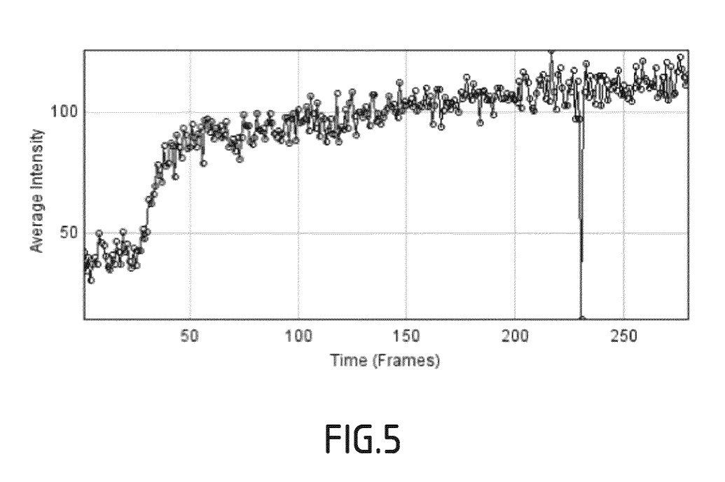

An example of image is illustrated by FIG. 3, on which Z1 corresponds to the lung, Z2 to the liver and P to a point of the diaphragm.

At the end of the step of providing S50, a set of images is obtained.

At the step of determining S60, the position of the diaphragm of the subject is determined.

The step of determining S60 is carried out for each image of the initial set.

According to the example of FIG. 2, the step of determining comprises several operations: a first operation of calculating, a second operation of obtaining and a third operation of deducing

At the first operation of calculating, it is calculated a spatial derivative of the signal intensity along at least one detection line.

A detection line is a line which crosses an area of an image along which at least one point is believed to belong to the diaphragm.

A detection line is, for instance, a line which links together a point of the liver and a point of the lung.

For instance, the spatial derivative is calculated by applying a gradient along the detection line.

At the end of the first operation, it is obtained a calculated derivative over the detection line. Such calculated derivative is called a profile.

At the second operation of obtaining, the position of the extremum of the derivative (profile) is obtained.

According to a specific embodiment, the position of the extremum is obtained by calculating the position for which the derivative of the spatial derivative of the signal intensity along the detection line is equal to 0.

According to another embodiment, the extremum is only searched for the positions for which the spatial derivative is superior to a given threshold.

At the end of the second operation, it is obtained the position of the extremum for each detection line.

At the third operation of deducing, the position of a point belonging to the diaphragm is deduced.

Such operation of deducing is based on the obtained extremum.

In a simple embodiment, the position of a point is the position of each extremum obtained at the end of the second operation.

In a more elaborated embodiment, the position is calculated based on several positions of extremum.

According to a specific example, the calculating operation is achieved for at least 10 detection lines of one image.

In such example, the position may be an average of the 10 positions of extremum.

In such case, the point belonging to the diaphragm is a specific point, such as the center of the diaphragm.

In another embodiment, the mean of the ten profiles is calculated to obtain a mean profile and the position is the position of the extremum of the mean profile.

At the end of the third operation, it is obtained a position of a point belonging to the diaphragm.

In complement or alternatively, the calculating and obtaining operations are carried out for several distinct detection lines, to obtain several positions, the deducing operation being carried out by calculating an average of the obtained positions.

According to a specific embodiment, a craniocaudal axis can be defined for the subject.

The craniocaudal axis is defined from the cranial to the caudal end of the subject.

More precisely, the craniocaudal axis is included in the sagittal plane (that is the median plane) of a subject.

In addition, the craniocaudal axis is vertical when the subject is standing up on a ground.

In other words, on the examination table of an MRI imager, the craniocaudal axis is horizontal when the subject relies on the examination table.

In such specific embodiment, the angle between each detection lines and the craniocaudal axis is inferior to 5°, preferably inferior to 2° and more preferably inferior to 1°.

According to another embodiment, the or each detection line extending on each image in a direction parallel to the craniocaudal axis.

At the end of the step of determining S60, the position of the diaphragm is obtained for each image of the initial set.

According to the embodiments, the position of the diaphragm refers to different physical realities. For instance, the position of the diaphragm is the position of the specific point, such as the center of the diaphragm. In variant, the position of the diaphragm is a set of points.

In any case, this position is named the determined position in the remainder of the specification.

At the step of choosing S70, a reference image in the initial set is chosen.

According to a first embodiment, the choice is of no importance. The image can, for instance, be chosen in a random way.

For instance, the image is the first one of the set of images or the last one of the set of images.

According to a second embodiment, the choice of the image is based on an analysis of the quality of the image. For instance, the image is chosen if a specific criterion is fulfilled. Such criterion may be a criterion relative to a signal-to-noise ratio for the image. The reference image is, in this context, an image for which the signal-to-noise ratio is superior or equal to a threshold.

The determined position for the reference image is named the reference position.

At the step of compensating S80, the difference between the determined position and the reference position is compensated.

The step of compensating S80 is carried out for each image of the initial set.

By comparison with the reference position, the offset, thus the rigid motion in the craniocaudal axis, is quantified.

Such offset is calculated by applying a difference between the determined position and the reference position for each image.

Provided the reference image is the first image, the calculation of the offset for each image leads to obtain a temporal evolution of the offset, which is labeled φ(t).

Then, the step of compensating comprises applying a translation corresponding to the offset between the determined position and the reference position.

The applied translation is specific to each image of the set of images.

One way for applying such translation is to use a circular permutation.

As an example, in the case the reference image is the first image, the following formulas can be used:

$${F}_{ϕ} ( {a}_{i} ) = \{ \begin{matrix}{a}_{i , t} + ϕ ( t ) when i + ϕ \leq n & \\ {a}_{i , t} + ϕ ( t ) - n when i + ϕ \geq n & \end{matrix}$$

Where:
F_{φ} denotes the transformation applied to a non null set of voxel(s) aᵢ, (typically a column of voxels) in a given direction by φ.
n is the length of the pixel set (in voxels),
t is the time and
φ(t) is the offset apply to the set of voxel according to the time (in voxel).

A voxel represents a value on a regular grid in three-dimensional space. In such context, a voxel is the temporal evolution of a given pixels.

Such specific formulas are thus a specific example of applying a circular permutation.

At the end of the step of compensating S80, a corrected set of images is obtained.

The results obtained are notably illustrated by the comparison of FIGS. 4 and 5.

FIG. 4 is a graph showing the evolution of the intensity on point P with time (that is to say with images) without carrying out the method for post-processing whereas FIG. 5 is a graph showing the evolution of the intensity on point P with time (that is to say with images) when carrying out the method for post-processing.

The fact that the evolution of intensity corresponds to a smoother curve indicates that the compensation of the respiratory motion has been achieved.

Such method enables to compensate for the translation component of the respiratory motion.

Only compensating the translation component corresponds to consider that the respiratory motion is only a translation and thus neglecting the rotation and the change of volume during the respiratory motion.

For most application, such compensation is sufficient to enable further analysis of the images.

Another advantage of the method is the absence of breath-holding requirement during dynamic acquisition since ghosting artifact was importantly reduced by the use of a key-hole acquisition with stochastic trajectories for k-space filling and misregistration between 2D+t frames were compensated by the retrospective respiratory motion correction including in our post-processing pipeline. In this regard, rather than to use a more conventional automatic registration algorithm we develop and include a dedicated algorithm in the reconstruction pipeline. The rationale behind this choice was that the functions of similarity used by automatic registration algorithms are sensitive to pixel intensity variation according to the time. Therefore, dynamic contrast enhancement confounds pixel intensity variations linked to the motion and induces substantial registration errors, particularly during the perfusion phase where signal intensity variations over the time are the most important. Amer-based semi-automatic methods could be an alternative to iconic, nevertheless, their use are limited by the prohibitive number of dynamics. Nevertheless, this method did not accounting for the non-rigid component of the motion and can be only used for coronal plane acquisitions

In addition, the method relies on the fact that there is an apparent contrast between the lung and the liver. Indeed, the lung appears to be a dark pixel in the image while the liver appears as a clear pixel, notably if the liver is subjected to a disease.

Such contrast is improved by the use of a median filter applied on the image, such filter tending to eliminate the salt-and-pepper noise.

The salt-and-pepper noise is also named Fat-tail distributed or “impulsive” noise or spike noise. An image containing salt-and-pepper noise will have dark pixels in bright regions and bright pixels in dark regions. This type of noise can be caused by analog-to-digital converter errors, bit errors in transmission.

For instance, between the step S50 of providing and the step S60 of determining, a median filter step is applied.

As a specific example, the median filter is a Gaussian filtering using a kernel of 3 pixels by 3 pixels.

More generally, the method for post-processing applies to any quantitative imaging technique.

For instance, the quantitative imaging technique is a CT imaging.

A CT scan, also called X-ray computed tomography (X-ray CT) and computerized axial tomography scan (CAT scan), makes use of computer-processed combinations of many X-ray images taken from different angles to produce cross-sectional (tomographic) images (virtual “slices”) of specific areas of a scanned object, allowing the user to see inside the object without cutting.

It is also to be noted that the method has been illustrated to thoracic diaphragm. However, such method could easily be transposed to urogenital diaphragm or pelvic diaphragm.

The embodiments and alternative embodiments considered here-above can be combined to generate further embodiments of the invention.

1-16. (canceled)

17. A method for post-processing a set of images, the method comprising at least the step of:
providing an initial set of images, the initial set of images comprising a plurality of images of a region of interest of a subject having a diaphragm, the images defining a field of view, the field of view including the region of interest and a part of the diaphragm of the subject,
for each image of the initial set, determining the position of the diaphragm of the subject to obtain a determined position for the considered image,
choosing a reference image in the initial set, the determined position for the reference image being a reference position, and
for each image of the initial set, compensating the difference between the determined position and the reference position to obtain a corrected set of images.

18. The method according to claim 17, wherein the field of view includes the whole diaphragm.

19. The method according to claim 17, wherein the field of view includes a portion of at least one fourth of the lung.

20. The method according to claim 17, wherein the region of interest is the liver.

21. The method according to claim 17, wherein each image is a map of a signal intensity on the field of view, the step of determining comprising the operation of:
calculating a spatial derivative of the signal intensity along at least one detection line, to obtained a calculated derivative,
obtaining the position of the extremum of the derivative,
deducing the position of a point belonging to the diaphragm based on the obtained extremum.

22. The method according to claim 21, wherein the calculating operation is achieved for at least 10 detection lines of one image.

23. The method according to claim 21, wherein a craniocaudal axis is defined for the subject, the or each detection line extending on each image in a direction parallel to the craniocaudal axis.

24. The method according to claim 21, wherein the calculating and obtaining operations are carried out for several distinct detection lines, to obtain several positions, the deducing operation being carried out by calculating an average of the obtained positions.

25. The method according to claim 17, wherein the compensating step is achieved by using a circular permutation.

26. The method according to claim 25, wherein each image comprises voxels, the circular permutation being applied to a set of non null voxels arranged along a given direction.

27. The method according to claim 17, wherein the time interval between two images of the initial set of images is comprised between 2 seconds and 5 seconds.

28. The method according to claim 17, wherein the images have been acquired with a quantitative imaging technique.

29. The method according to claim 28, the quantitative imaging technique is a magnetic resonance imaging technique.

30. The method according to claim 29, wherein the method comprises a step of injecting a contrast agent before the acquisition of the initial set of images.

31. A non-transitory computer-readable medium on which is stored a computer program product comprising instructions for carrying out the steps of a method according to claim 17 when said computer program product is executed on a suitable computer device.
